(19) United States
(12) Patent Application Publication
(10) Pub. No.: US 2005/0282145 A1
(43) Pub. Date: Dec. 22, 2005

(54) Method for identifying anti-neoplastic compounds

(76) Inventor: Wen Luan Wendy Hsiao, Kowloon (HK)

Correspondence Address: KNOBBE MARTENS OLSON & BEAR LLP, 2040 MAIN STREET, FOURTEENTH FLOOR, IRVINE, CA, 92614, US

(21) Appl. No.: 10/870,284

(22) Filed: Jun. 16, 2004

(51) Int. Cl. C12Q 1/00

(52) U.S. Cl. 435/4

(57) ABSTRACT

Aspects of the present invention relate to methods of evaluating substances for their effects on cellular proliferation. Particular aspects of the invention use cultures of cells containing more than one type of cell.

### BACKGROUND OF THE INVENTION

1. Field of the Invention

Aspects of the present invention relate to methods for detecting compounds that antagonize the growth of malignant cells.

2. Description of the Related Art

The anti-tumor effect of mushrooms has long been observed in Asia, especially in China and Japan. The analysis of various species of mushrooms has resulted in the identification of extracts containing a family of high molecular weight, hot-water-soluble polysaccharides (PS) and polysaccharide-peptide complexes (PSPC) which have tested positive for anti-tumor activities in animal studies (1). NMR analysis reveals that the anti-tumor polysaccharides are composed of a variety of linear and branched glucans. They appear in various conformations, and some are in the gel state. Due to the complexity and heterogeneity of PS and PSPC and limited suitable bioassay systems, the mechanism of the action of these polysaccharides remain obscure. Until now, research on the anti-tumor activities are mainly based on the results derived from experiments with implanted Sarcoma180 or chemically induced tumors in mice (2-6). Antimitotic tests in tumor cell lines have been, so far, rather inconsistent or negative (1,2,4,7-9). Aside from their anti-tumor effects, mushroom-derived PS and PSPC seem to function as immunomodulators; this was observed in animals as well as in cultured macrophages and T-lymphocytes.

References which are cited in the present disclosure are not necessarily prior art and therefore their citation does not constitute an admission that such references are prior art in any jurisdiction. All reference materials cited in this application are hereby incorporated by reference in their entirety.

In order to gain a more comprehensive understanding of the anti-cancer effects of these extracts, it is necessary to investigate the anti-tumor activity of mushrooms with novel approaches.

### SUMMARY OF THE INVENTION

One aspect of the invention features a method for evaluating the ability of a substance to influence the rate of cellular proliferation that comprises exposing a culture comprising transformed cells and non-transformed cells to said substance and determining whether said substance influences the proliferation rate of said transformed cells. In preferred embodiments of the invention, the rate of cellular proliferation is measured in terms of a parameter selected from the group consisting of reduction in foci, reduction in number of cells, number of apoptotic cells, number of necrotic cells, measures of cell quiescence, changes in the intensity of a signal and the presence of a marker. The substance may comprise biological extracts, isolates, fractions, compounds, proteins, peptides, glucans, polysaccharides, polysaccharide protein complexes and/or inorganic compounds. Additional embodiments feature transformed and non-transformed cells that differ based on a characteristic selected from the group consisting of organism of origin, organ of origin, tissue or origin, clonal line, pre-experimental treatment, transfection with plasmid, exposure to a microorganism, infection with a virus and exposure to ionizing radiation. In some embodiments, the step of determining whether the substance influences the proliferation rate of the transformed cells comprises comparing the proliferation rate of said transformed cells in said culture to the proliferation rate of transformed cells in a control culture. In particular embodiments, said control culture comprises transformed cells and nontransformed cells which have not been contacted with said substance. Additional aspects of the invention can include control cultures comprising transformed cells which have not been contacted with said substance. In additional embodiments of the invention, said step of determining whether said substance influences the proliferation rate of said cells comprises determining whether said substance decreases the proliferation rate of said cells. Some embodiments of the invention feature cultures comprising transformed cells and nontransformed cells is obtained by mixing transformed cells with nontransformed cells.

Additional embodiments of the invention feature methods for evaluating the ability of a substance to influence the rate of cellular proliferation comprising contacting a culture comprising transformed cells with said substance and with culture medium from a culture comprising nontransformed cells and determining whether said substance influences the proliferation rate of said transformed cells. In some embodiments of the invention, transformed cells comprise a sequence that increases the proliferation rate of the cells, said sequence having been introduced to the cells by a method selected from the group consisting of stable transfection, transient transfection, random recombination, homologous recombination, exposure to a viral vector, exposure to ionizing radiation and chemical mutation. In some embodiments of the invention, transformed cells comprise sequence for expression of a marker molecule that can be used to distinguish said transformed cells from said nontransformed cells. Additional aspects of the invention include markers that indicate expression of a protein, cell replication or release of compound into the media. In some embodiments, the cell culture comprises fibroblast cells. In additional embodiments of the invention, the rate of cellular proliferation is measured in terms of a parameter selected from the group consisting of reduction in foci, reduction in number of cells, number of apoptotic cells, number of necrotic cells, measures of cell quiescence, changes in the intensity of a signal and the presence of a marker. Particular embodiments feature methods wherein the substance is selected from the group consisting of biological extracts, isolates, fractions, compounds, proteins, peptides, glucans, polysaccharides, polysaccharide protein complexes and inorganic compounds. Additional embodiments also feature methods wherein said transformed and said nontransformed cells are cultured in the same media, separated by a permeable barrier or membrane.

Another aspect of the invention features biologically active molecules that reduce the proliferation of transformed cells in culture with non-transformed cells and that is isolated from Tricholoma lobayense by a process comprising boiling a culture of Tricholoma lobayense in water, centrifuging to remove insoluble material, binding of soluble material to an ion-exchange chromatography column, elution of the soluble material from the column and isolating an elution fraction containing said molecule(s).

### BRIEF DESCRIPTION OF THE DRAWINGS

FIG. 1 shows an image of a giemsa stain of transformed foci in R6 cells cultures transfected with pT24 plasmid DNA. Transfected cultures were grown in normal medium alone (A); normal medium with 100 μg/ml DEAE column-bound fraction A2 (i.e. PSPC-enriched fraction)(B); or normal medium with 100 μg/ml DEAE column-unbound fraction A1 (C) of Tricholoma filtrates. Experiments were carried out as described in Table 3

FIG. 2 shows conditions for time course studies of the effect of PSPC-enriched fraction A2 of Tricholoma filtrate on the number of transformed foci in R6 cultures transfected (at day 0) with pT24 plasmid DNA. Where indicated, transfected cultures were treated with 100 μg/ml of A2 fraction of Tricholoma filtrate described in Table 3. Experiments were terminated at day 20, and stained with Giemsa stain for scoring. Relative number of foci is the ratio of foci obtained in the presence of drug to that in the absence of drug (i.e. no treatment control).

FIG. 3 shows the effects of the PSPC-enriched fraction A2 of Tricholoma filtrate on colony formation of normal and transformed R6 cells. Panels A & B: 500 of R6 or R6/GFP-Ras-transformed cells per 90 mm plate were seeded in DMEM plus D10CS in the presence and absence of Tricholoma filtrate A2 fraction (200 μg/ml); Panel C: 500 R6/GFP-Ras cells were seeded on a lawn of 2.5×10⁵ normal R6 cells plated in 90 mm plate 24 h earlier. The Tricholoma filtrate A2 fraction (200 μg/ml) was then added to the co-cultures of normal and transformed R6 cells grown in D5CS 24 hr after the seeding of the transformed cells. At the end of 2 weeks, culture plates were fixed with 10% formaldehyde, stained with Giemsa stain and photographed. GFP-Ras-colonies derived from the co-cultures were viewed and photographed under a fluorescent microscope at 100 and 400× magnifications.

FIG. 4 shows the effects of the PSPC-enriched fraction A2 of Tricholoma filtrate on Ras protein expression in normal and transformed Rat 6 cells. 2.5×10⁵ R6 or R6/GFP-Ras-transformed cells per 90 mm plate were seeded in DMEM plus D10CS. Cultures were fed the following day with fresh medium in the presence (+) and absence (−) of Tricholoma filtrate (A2) (200 μg/ml) and then fed twice a week. At the end of 14 days, cells were washed with cold PBS, lysed in NET buffer plus protease inhibitors and collected for western blot analysis as described in the Materials and Methods. Protein extracts obtained from the treated and untreated R6 and R6/GFP-Ras cultures were loaded 40 μg per lane and separated on 12% PAGE gel by electrophoresis. The resulting blot was hybridized sequentially with anti-Ras (Santa Cutz), anti-GFP (Clontech), and anti-actin (Santa Cutz) antibodies and visualized using Amersham ECL Western Blotting Detection Kit according to the manufacture manual.

FIG. 5 displays the effects of the PSPC-enriched fraction A2 of Tricholoma filtrate on the subcellular localization of GFP-Ras protein in GFP-ras-transformed R6 cells co-cultivated with normal R6 cells. The co-cultures were prepared as described in FIG. 3 legend, and were treated (+) or untreated (−) with Tricholoma filtrate (200 μg/ml) for 7 days. Panels A-C are fluorescent microscopic views of three individual R6/GFP-Ras colonies derived from the untreated co-cultures, while Panels D-F are three individual colonies derived from the treated co-cultures.

FIG. 6 shows colony formations of R6/GFP-ras transformed cells co-cultured with normal R6 cells in medium with or without total saponins extracted from Gynostemma pentaphyllum (Gp). The transformed cells (10³) were seeded on plates either with or without 5×10⁵ R6 cells that had been pre-seeded one day earlier. Cells were fed with medium that either contained (+Gp) or did not contain (−Gp) of Gp saponins. The cultures were maintained for 12 days, then stained with Giemsa stain.

### DETAILED DESCRIPTION OF THE PREFERRED EMBODIMENT

The anti-tumor effects of PS and PSPC of higher fungi have long been investigated in tissue cultures, animal models and clinical patients, yet the mechanisms underlying the activity of PS and PSPC are unclear. Some evidence suggests that the anti-tumor effect of PS may be mediated through the cytokines released by activated macrophages and T-lymphocytes, instead of through direct cytocidal effects on tumor cells (4,8-11).

Compounds that show anti-cancer effects may be acting in a wide variety of ways to exert those effects. It is useful to have systems for studying the effects of compounds on separate elements that contribute to the transformation of cells. To this end, in a previous study we wished to examine the effects of PS and PSPC on cells transformed by virtue of the transfection of the ras oncogene. We developed a simple Rat 6 (R6) cell system by which the inhibitory effects of non-cytotoxic chemicals can be assessed by focus formation assay upon transfection of ras oncogene to the host cells. Using this system, two well studied medicinal mushrooms with anti-cancer potential, Ganoderma lucidum and Tricholoma lobayense, were examined for their possible adverse effects on cell transformation induced by ras oncogene. Results indicated that both species of mushrooms yielded strong inhibitory effects on ras-induced cell transformation. Further study on T. lobayense indicated that the DEAE-column-bound, polysaccharides-peptide enriched fraction, but not the unbound fraction, showed strong inhibition in a dosage dependent manner. Subsequent time course studies revealed that the continued presence of the extract in the transfected cultures was required for a maximum inhibitory effect. At the same time, we also observed that significant level of inhibition occurred even when the application of the extract was delayed until day 12 after transfection.

In one embodiment of the present invention, in order to further investigate the anti-cancer properties of PS and PSPC, a cell line expressing a tagged ras-fusion protein was developed, R6/GFP-Ras. This stable transformed cell line, which expresses green fluorescent protein-ras fusion protein, was used to create a co-culture assay with normal R6 cells. We demonstrated that R6/GFP-Ras cells grew into green fluorescent foci with striking transformed morphology in the absence of extracts. However, in the presence of extracts, R6/GFP-Ras cells, in most cases, remained as small colonies compiled with only few green fluorescent cells. Moreover, the inhibitory effect requires the presence of untransformed R6 cells. In our study, mushroom extracts have no effect on the growth of individually cultured normal and transformed R6 cells.

One aspect of the invention features a method for examining substances, mixtures or compounds for effects on proliferative activity of cells. In a preferred embodiment, the cell sample used in the method contains a mixture of cells. In an even more preferred embodiment, the mixture of cells is a co-culture that contains both cells in which a sequence which increases the rate of cellular proliferation or which overcomes contact inhibition, such as an oncogene sequence, is present and cells in which the sequence is not present. A sequence which increases the rate of cellular proliferation can be introduced by stable or transient transfection, recombination (random or homologous), viral vector, ionizing radiation, chemical mutation or other methods familiar to those with skill in the art. Alternatively, the sequence may be present as the result of a spotaneous mutation such as a mutation which gives rise to a tumor. Such cells are referred to herein as “transformed cells”. Thus, the term “transformed cells” includes, but is not limited to, cells which have been transfected with an oncogene, cells which are immortal, cells which show a reduced level of contact inhibition or cells which are malignant. Some embodiments of the invention feature cell mixtures containing more than transformed and untransformed cells. In embodiments of the invention comprising a mixture of transformed and untransformed cells, the ratio of one type of cell to the other type of cell may be anywhere from about 1:1 to 1:10, 1:20, 1:30, 1:40, 1:50, 1:100, 1:1000, 1:2000, 1:5000 or 1:10,000. Preferably, the transformed and non-transformed cells are identical except for the presence of the sequence which increases the rate of cellular proliferation or which overcomes contact inhibition. For example, cells of a particular cell line can be mixed with samples of cells from the same cell line that have been transfected with expression plasmids. Some embodiments of the invention feature cells that have been exposed to chemical compounds, biological agents, ionizing radiation, forms of non-ionizing energy or physical treatments before or during their use in the methods of the invention. In other embodiments, the transformed and non-transformed cells originate from different cell lines, different cell types, different tissues, different organs and/or different organisms. In some embodiments, cells of both types are fibroblast cells. In additional embodiments, one type of cell used comprises fibroblast cells. Additional cells types that may be used with particular embodiments of the invention include, but are not limited to, human bladder tumor cell lines, such as T24 cells. One example of an embodiment of a method of the invention is given below.

In one example of an embodiment of the invention, using a stable transformed cell line of R6 cells that express green fluorescent protein-ras fusion protein (R6/GFP-Ras) in a co-culture assay with normal R6 cells, we demonstrated that R6/GFP-Ras cells grew into green fluorescent foci with striking transforming morphology in the absence of extracts. However, in presence of extracts, we found that in most cases R6/GFP-Ras cells remained as small colonies compiled with only few green fluorescent cells. Moreover, the inhibitory effect requires the presence of R6 cells. In our study, mushroom extracts have no effect on the growth of individually cultured normal and transformed R6 cells. It is noteworthy that the extracts do not affect the level, or the subcellular localization of the Ras protein. Collectively, the data produced by an embodiment of the invention strongly suggest that the inhibitory effect of the mushroom extracts is not due to a direct killing of the transformed cells, rather, it may be mediated through the surrounding normal R6. Our data provides evidence for a novel alternative mechanism in which the mushroom polysaccharide peptides exert anti-cancer effect by targeting the ras-mediated signaling pathway.

Some embodiments of the invention feature a method for examining substances for effects on the proliferative rate of cells using transformed and untransformed cells. In some embodiments of the invention, the substance is present as an extract mixture or as a fraction of an extract mixture that has been isolated from other fractions of an extract mixture in some fashion. In some embodiments, the substance is a compound. In some embodiments of the invention, the transformed and non-transformed cells are not co-cultured. For example, in some embodiments, a substance can be tested for its abilities to enhance or inhibit cellular proliferative and/or tumorgenic activity by contacting transformed cells with the substance and with culture medium for a culture of normal cells treated with test materials. The culture medium from the normal cells may be placed in cantact with the tranformed cells prior to, simultaneously with, or after the tranformed cells are contacted with the substance. In additional embodiments, the culture media from the culture of non-transformed cells is processed in some manner before addition to the culture of transformed cells. In some embodiments, the processing features filtration, centrifugation and/or additional processes to separate the culture media from the non-transformed cells into fractions differing in content. Any or all of these fractions may then be applied to the transformed cells and the proliferative activity of the transformed cells is evaluated to determine whether the substance influences the proliferative activity of the transformed cells. Some embodiments of the invention feature multiple cultures of non-transformed cells from which media samples are removed and added to the transformed cells. In some of these embodiments, media samples or fractions from two or more cultures of non-transformed cells are added to a culture of transformed cells. In other embodiments, media samples or fractions from one or more cultures of non-transformed cells are added to more than one culture of transformed cells. In a particular embodiment, fractions of a media sample from a culture of non-transformed cells are divided up and added to cell cultures of transformed cells, allowing for a comparison of the effects of different media fractions or different amounts of the same fraction. Additional embodiments feature a variety of cells and cell types for use in a culture comprising transformed and non-transformed cells. These cells and cell types feature a wide variety of normal and transfected cells, malignant cells, cells in which viruses are actively replicating, cells which are infected with virus but are not actively producing virus and/or cells which express individual oncogenes, tumors suppressor genes, DNA repair genes, viral or host proteins.

An additional embodiment of the invention features co-culturing of non-transformed cells and transformed cells without contact or co-mingling of these different types of cells. Persons with skill in the art are familiar with cell culture inserts, membranes and transwell plate systems which allow the division of a single plate or well into separate sections but allow for media components to diffuse between both chambers. For example, polycarbonate membranes are commercially available with a range of pore sizes for use in a wide variety of experimental settings. Such membranes allow for co-culturing of cell populations in separate compartments, keeping different cell types growing in the same media physically separated from one another.

In an additional embodiment of the invention, a method for examining the activity of substances, mixtures or compounds for effects on cell proliferation uses at least one cell type that expresses a marker or tag. Preferably, the transformed cells express a marker and/or tag which can be used to measure the extent of their proliferation. The marker and/or tag may be of a variety of types, including but not limited to fluorescent markers, affinity markers and peptide moieties or tags. Individuals with skill in the art are familiar with a variety of different marker and tag molecules. In some embodiments, expression of a marker and/or tag would be indicative of the expression of a particular protein or type of protein. For example, in an embodiment of the invention, the expression of a fluorescent marker indicates that an oncogene is being expressed in that cells. Additional embodiments feature cells that express markers and/or tags in response to, in tandem with or in advance of a variety of cellular activities and conditions, including increasing proliferative activity or decreasing proliferative activity. Examples of peptide tags that may be used with embodiments of the invention are portions of the influenza hemaglutinin (HA) protein, portions of maltose binding protein (MBP) and polyhistidine tags. Some embodiments feature the examination of cell populations using antibodies, monoclonal or polyclonal, to any of a variety of targets, including but not limited to peptide tags, any number of CD (cluster designation) molecules, and peptides expressed from exogenous sequences. Some embodiments feature the examination of cell populations with a variety of antibodies directed at secreted, transmembrane and cytosolic proteins, both originating from endogenous and exogenous genetic sequences, including cytokines and growth factors. Some embodiments feature the use of fluorescent markers such as domains of Green Fluorescent Protein (GFP) and Red Fluorescent Protein (RFP). Other derivatives and functional equivalents of domains of Green Fluorescent Protein (GFP) and Red Fluorescent Protein (RFP) may also be used to practice some embodiments of the present invention.

Some embodiments of the present invention feature methods for screening potential anti-tumor drugs. In some embodiments, potential anti-tumor drugs that have an indirect effect on the tumor/transformed cells are screened. In some embodiments, potential anti-tumor drugs stimulate non-transformed cells to secrete anti-tumor substances and/or act on adjacent transformed cells.

Embodiments of the present invention feature co-cultures of two or more different cell types comprising transformed and non-transformed cells. Additional embodiments feature the use of two or more cultures of cells comprising transformed and non-transformed cells. As used herein, “cell type” refers to any classification of a cell or a line of cells, including the original organism, organ, tissue type or cell type from which the cell or cell line was developed. Cells of different “cell types” can also be cells that originated from the same cell or cell line and subsequently became differentiated into categories that are distinguishable under some conditions. Differentiation of cells into different types can be via physical, chemical or biological means. Some means of differentiating cells in separate cell types include, but are not limited to, stable transfection, transient transfection, random recombination, homologous recombination, exposure to a viral vector, exposure to ionizing radiation and chemical mutation.

As used in the present specification and claims, the terms “comprise,” “comprises,” and “comprising” mean “including, but not necessarily limited to.” For example, a method, apparatus, molecule or other item which contains A, B, and C may be accurately said to comprise A and B. Likewise, a method, apparatus, molecule or other item which “comprises A and B” may include any number of additional steps, components, atoms or other items as well.

### EXAMPLES

### Example 1

### Testing of Mushroom Extracts with Co-Cultured Cell Populations

1. Introduction

It has previously been demonstrated that the established rodent cell line R6 is resistant to transformation induced by a potent c-H-ras (T24) oncogene in a focus formation assay. The transforming efficiency of T24, however, can be modulated by treatment with various tumor promoters and factors (12,13). Using this R6/ras assay system the inhibitory or enhancing effects of various chemicals on T24 induced-transformation has been assessed (14-16). In the current study, we explored the anti-tumor activity of extracts of mushrooms using the focus formation assay built around the R6/ras model system.

In the study, we focused on two medicinal mushrooms: Ganoderma lucidum (G. lucidum) and Tricholoma lobayense (T lobayense). Both mushrooms exhibit anti-tumor activities, based mainly on animal studies (1-4, 17). T. lobayense is a native Hong Kong species. G. lucidum is an important traditional medicine in China and Japan, used for promoting health and treatment of various diseases, including cancer. Our results showed that ras-induced transformed foci were effectively inhibited by the addition of extracts of G. lucidum and T. lobayense in a dosage dependent and time dependent manners. Data also revealed that the PS fraction of T. lobayense would only exert inhibitory effect to Ras-transformed cells when cells were co-cultivated with normal R6 cells, suggesting a novel mechanism in which the inhibitory effect of PS is mediated through the surrounding normal R6 cells.

2. Materials and Methods

2.1 Preparations of Mushroom Samples

Fruiting bodies of G. lucidum were homogenized and extracted with boiling distilled water for 6 hr to obtain the PS-enriched preparations. After centrifugation to remove the insoluble portion, the water soluble extracts were lyophilized, then kept at room temperture for later usage. Liquid mycelium cultures of G. lucidum were also used to obtain PS-extract. Prior to extraction, the cultures were filtered and precipitated with ethanol according to Liu et al, 1995 (17). The precipitates were dissolved in distilled water, centrifuged to remove the insoluble, then lyophilized and designated as mycelium filtrate. Tricholoma lobayense was originally isolated and established in cultures by S. T. Chang's Laboratory at the Chinese University of Hong Kong, Hong Kong. T lobayense. was cultured in nutrient broth as described (17). Liquid cultures containing the secreted fungal PS was prepared as above. The water-soluble, PS-enriched components were lyophilized and designated as mycelium filtrate. Part of the filtrate was further fractionated into the unbound Fraction A1 and the salt-eluted bound Fraction A2 using a DEAE-cellulose ion exchange chromatography column (17). Both fractions were dialyzed against ddH₂O and lyophilized for later usage.

2.2 Cell Cultures and Plasmids

The rat 6 (R6) cell line was a subclone of Fisher rat embryo fibroblasts originating from the Freeman Laboratory (18). The R6/T24 cell line is a clonal R6 cell line transfected by the activated human c-Ha-ras oncogene (12). R6/GFP-Ras cell line is a transformed clonal cell line established from a transformed focus derived from R6 cultures transfected by a GFP-ras fusion vector in our lab. Cells were grown in Dulbecco modified Eagle medium (DMEM) supplemented with 10% calf serum (D10CS)(Gibco). Cultures were maintained in a humidified incubator at 37° C. with 5% CO₂ in air and fed twice a week with fresh medium. Plasmid pT24 contains a 6.4 kb BamHI fragment corresponding to the coding sequence of the human bladder c-Ha-ras oncogene. The plasmid pT24 was obtained from M. Wigler's Laboratory.

2.3 Focus Formation Assay

The standard focus formation assay and treatment of the cultures were performed as described earlier (12). In brief, 5×10⁵ cells seeded in 90 mm plate were transfected with 1 μg T24 plasmid DNA and 20 μg R6 genomic DNA as carrier DNA by DNA-mediated transfection procedure based on Bacchetti and Graham (19) and Wigler et al. (20), with slight modifications (12,13). To determine the effects of mushroom extracts on ras-induced focus formation, transfected cultures were fed with DMEM plus 5% fetal calf serum (D5FCS) in the presence and absence of test sample on day 2 upon transfection, then continued feeding with each respective growth medium twice a week throughout the experiment. Lyophilized samples of mushroom preparations were weighted, dissolved in boiling ddH₂O, then centrifuged at 10,000 rpm for 20′ to remove residues. The supernatant was sterile filtered, then added to the growth medium at designated concentrations. All experiments were performed in six replicate plates.

2.4 Cytotoxicity Assay

Assays of the cytotoxic effects of each sample were performed on both R6 and R6/T24 cell lines. Cells were seeded in triplicate at 10⁴/60 mm plate in D10CS. The next day, test samples were added to culture media and incubated for 5 days. At the end of treatments, cells were trypsinized and counted using a Coulter Counter. Cytotoxicity was expressed as percent survival, i.e. cell counts of treated cultures divided by cell counts obtained from untreated cultures.

2.5 Colony Formation and Co-Culture Assays

For a better quantitative assessment of the inhibitory effect for drug testing, we designed a co-culture assay to simulate the focus formation assay. The assay was set up by seeding 500 ras-transformed R6 cells on 90 mm plates in triplicate that were pre-seeded with of 2.5×10⁵ of normal R6 cells 24 h earlier. A day after the seeding of the transformed R6 cells, the test sample was added to the co-cultures of normal and transformed R6 cells grown in DMEM plus 5% CS (D5CS). At the end of two weeks, culture plates were fixed with 10% formaldehyde, stained with Giemsa stain and photographed. In order to distinguish the transformed from the neighboring normal R6 cells, a transformed cell line, R6/GFP-Ras expressing green fluorescent GFP-Ras fusion protein was used in the co-culture assay. In a parallel experiment, the possible toxic effect of mushroom extracts on the growth of R6 and R6/GFP-Ras cells were tested by seeding 500 of each cell line separately in D10CS in the presence and absence of test chemical for 12 to 14 days. Cultures were stained and scored for total number of colonies per plate. All the experimental cultures were fed twice a week, either with or without the mushroom extract.

### 2.6 Western Blot Analysis

R6 or R6/GFP-Ras-transformed cells were seeded at a density of 2.5×10⁵ cells per 90 mm plate in DMEM plus D10CS. On the following day, cultures were fed with fresh medium in the presence or absence of the Tricholoma filtrate A2 fraction (500 μg/ml) and fed twice a week. Cells were washed with cold PBS three times and lysed in 400 μl NET buffer (150 mM NaCl, 50 mM Tris-HCl, pH 7.4, 5 mM EDTA, pH 8.0, 1 mM APMSF, 1 μM E-64, 1 μM pepstatin, 100 mM NaVO₅, and 10 μg/ml aprotinin) plus 1% NP-40 on ice according to Lu et al. (21). For western blotting analysis, 40 μg of protein extracts were loaded on a 12% SDS-PAGE gel. After separation, the proteins were transferred to a Hybond-C nylon membrane (Amersham), hybridized to either anti-Ras (Santa Cruz) or anti-GFP antibodies (Clontech) and visualized with the ECL detection kit (Amersham) according to the manufacture's manual. Blots were hybridized with anti-actin antibodies (Santa Cruz) to normalize the gel loading.

3. Results

3.1 Effect of Extracts of G. lucidum on Ras-Induced Transformation

In the primary experiment, extracts of fruiting bodies and filtrate of G. lucidum were tested. While both the fruiting bodies and mycelium filtrate were effective in inhibiting foci formation induced by c-Ha-ras oncogene, the latter seemed to be more effective (Table I). At 200 μg (dry weight)/ml and above (Table IB), mycelium filtrate showed nearly 100% reduction of foci. On the other hand, the fruiting body extract-treated cultures exhibited 56% inhibition at 200 μg/ml and 80% at a dose of 500 μg/ml (Table IA). Neither of the samples had a cytotoxic effect on either the host R6 or R6/T24 cells (Table II). Thus, the inhibitory effect of the mushroom extracts on ras-transformation is not due to a direct killing of cells transformed by ras oncogene in the focus formation assay.

TABLE I
Effect of hot water extracts of G. lucidum
on ras-induced transformation

| | | | Relative number |
|---|---|---|---|
| Material tested | Conc. (μg/ml) | Number of Foci | of foci* |

A. Extract of fruiting bodies

| Mock control | | 0 | |
|---|---|---|---|
| No treatment | 0 | 14.7 ± 4.0 | 1 |
| Extract | 10 | 13.7 ± 4.5 | 0.93 |
| | 100 | 8.3 ± 2.9 | 0.56 |
| | 500 | 3.0 ± 1.0 | 0.2 |
| | | | Relative number |
| Material tested | Conc. (μg/ml) | Number of Foci | of foci¹ |

B. Extract of mycelium filtrate

| | | | |
|---|---|---|---|
| Mock control | | 0 | |
| No treatment | 0 | 18.3 ± 2.3 | 1 |
| Extract | 100 | 2.7 ± 1.2 | 0.15 |
| | 200 | 1.0 ± 1.0 | 0.05 |
| | 500 | 1.3 ± 1.5 | 0.07 |

*Relative number of foci; the ratio of foci obtained in the presence of drug to that in the absence of drug (i.e. no treatment control).

TABLE II
Cytotoxicity of hot water extract of G. lucidum

| | Conc. | R6 cells | R6/T24 cells |
|---|---|---|---|
| Treatment* | (μg/ml) | % survival** | % survival** |
| No treatment | 0 | 100 | 100 |
| Fruiting body | 500 | 93 | 98 |
| Mycelium filtrate | 500 | 89 | 98 |

*The duration of the treatment was 5 days for all treatment groups.
**% survival = cell counts of treated cultures/cell counts of untreated cultures × 100%.

3.2 Effect of Tricholoma Filtrate on Ras-Induced Transformation

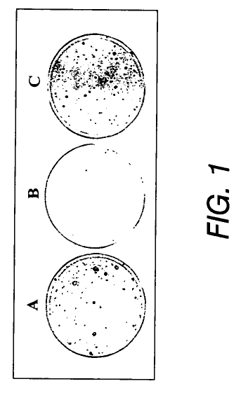

Both the total filtrate and the DEAE-column-bound fraction of T. lobayense filtrate were tested in R6 cells upon transfection of the ras oncogene. Data showed that the total filtrate and the DEAE-column-bound fraction A2 of T. lobayense markedly inhibited ras-foci, while the unbound fraction produced no effect (Table III & FIG. 1). The preparations had no or only slightly toxic effects on either normal or transformed R6 cells (Table IV). The nutrient broth used for the T. lobayense cultures alone presented no inhibitory effect.

TABLE III
Effect of extracts of Tricholoma filtrate on ras- induced transformation

| | | Relative number |
|---|---|---|
| Material tested | Conc. (μg/ml) | of foci |
| Mock control | — | 0 |
| No treatment | 0 | 1 |
| Total mycelium filtrate | 100 | 0.04 |
| | 200 | 0.10 |
| DEAE-column purified fraction | 100 | 0.04 |
| A2¹ | | |
| DEAE-unbound fraction A1¹ | 50 | 0.96 |
| | 100 | 0.91 |

¹The liquid cultures of T. lobayense was filtered, concentrated and precipitated with ethanol. The precipitate was dissolved in distilled water. The water soluble fraction was then applied to a DEAE-cellulose column. The unbound fraction A1 was eluted with distilled water, and the retained components (A2) were eluted with a salt gradient of NaCl (0 to 2 M) (5).

TABLE IV
Cytotoxicity of Extracts of Tricholoma filtrate

| | Conc. | R6 cells | R6/T24 cells |
|---|---|---|---|
| Treatment* | (μg/ml) | % survival | % survival |
| No treatment | 0 | 100 | 100 |
| Total filtrate | 400 | 98 | 91 |
| DEAE-column-bound | 100 | 80 | 93 |
| fraction A2 | | | |
| DEAE-column- | 100 | 101 | 105 |
| unbound fraction A1 | | | |

*The duration of the treatment was 5 days for all groups.

Dosage and Time Course Studies of Tricholoma Filtrate on Ras-Induced Transformation

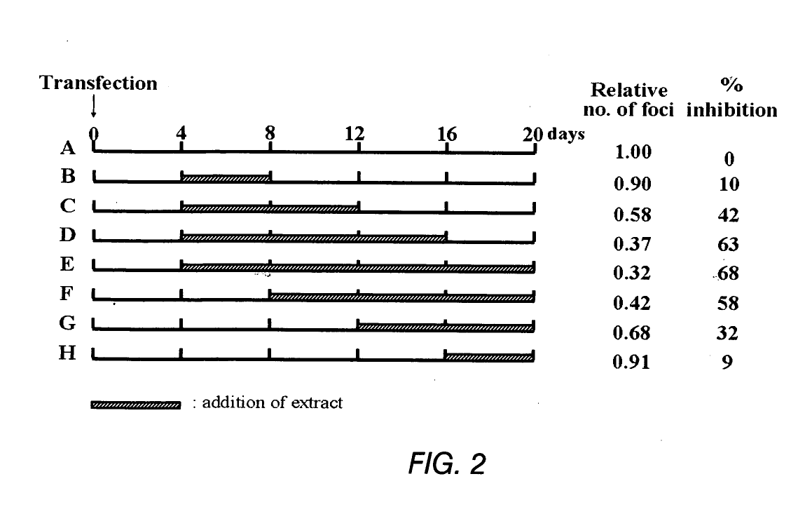

The inhibitory effect of the Tricholoma filtrate was further explored with regard to dosage and duration of treatment. Results revealed that the inhibitory effect of Tricholoma filtrate was dosage-dependent. Extracts in concentration as low as 1 μg/ml exerted a 19% inhibitory effect on the formation of ras-foci (Table V); Time course studies indicated that the maximal effect was obtained when transfected cultures were treated with Tricholoma filtrate from days 4 to 20 after the transfection. Interestingly, a 32% reduction in foci number was still obtained when the treatment was delayed until day 12 after the transfection of the ras oncogene (FIG. 2).

TABLE V
Dosage effect of DEAE-column purified fraction
of Tricholoma filtrate on ras-
induced transformation

| Test sample | Conc. (μg/ml) | Relative number of foci |
|---|---|---|
| Mock control (no T24 | 0 | 0 |
| DNA) | | |
| No treatment | 0 | 1 |
| DEAE-fraction A2* | 1 | 0.81 |
| | 5 | 0.78 |
| | 10 | 0.87 |
| | 20 | 0.56 |
| | 50 | 0.47 |
| | 100 | 0.32 |

*DEAE A2 fraction was prepared as described in Table III legend.

3.4 Effect of Tricholoma Filtrate on R6/GFP-Ras Cells Co-Cultivated with Normal R6 Cells

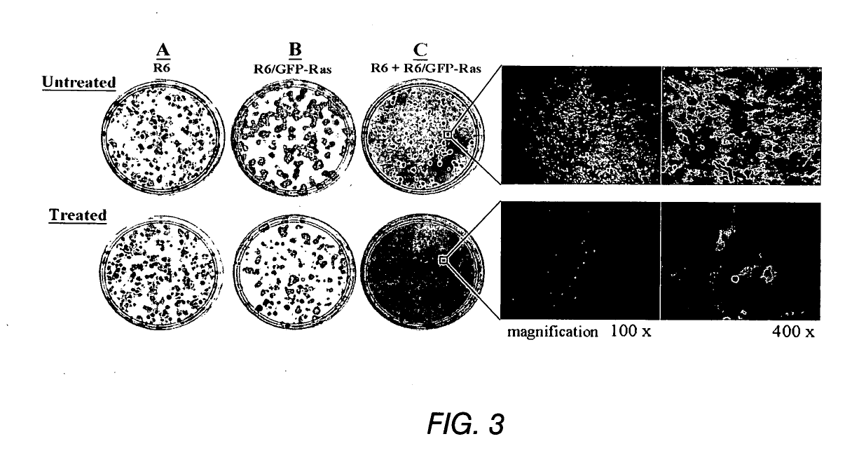

To further explore the nature of inhibitory effect of Tricholoma filtrate, we reconstituted the focus formation assay by seeding 500 R6/GFP-Ras cells on a 90 mm culture plate pre-seeded with 2.5×10⁵ normal R6 cells 24 hr earlier. The co-cultures were then maintained in D5CS medium in the presence and absence of Tricholoma filtrate. The effects of Tricholoma on colony formations of individual R6 and R6/GFP-Ras cell lines were compared to results of studies with the co-culture assay. Results indicated that addition of Tricholoma did not affect the colony formation (FIGS. 3A & B), nor the morphology of R6 or R6/GFP-Ras cells (data not shown). In the co-culture of R6 and R6/GFP-Ras (FIG. 3C), the R6/GPF-Ras cells formed many dense transformed colonies on the top of the monolayer of R6 cells in the absence of treatment, resembling the formation of transformed foci shown in FIG. 1. Addition of Tricholoma filtrate (200 μg/ml) effectively blocked the formation of the GFP-Ras transformed colonies. Indeed, under the fluorescent microscope, the growth of the green fluorescent colonies, representing the R6/GFP-Ras cells, was observed to be severely retarded in the presence of the Tricholoma filtrate, while the majority of the colonies grown in the absence of the drug treatment became sizable colonies (FIG. 3). It is important to note that treatment with Tricholoma does not affect the initial plating efficiency of R6/GFP-Ras cells as nearly the same number of GFP-positive cells were observed on both the treated and untreated plates under a fluorescent microscope, 24 hr upon the addition of the filtrate. Additional evidence came from the fact that early withdrawal of the treatment substantially reduces the inhibitory effect of the filtrate, resembling what we observed in the focus formation assay (data not shown).

3.5 Tricholoma Filtrate Posted no Effect on the Expression of GFP-Ras Protein

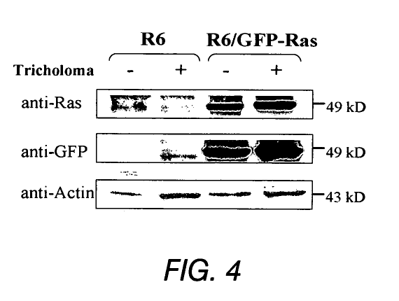

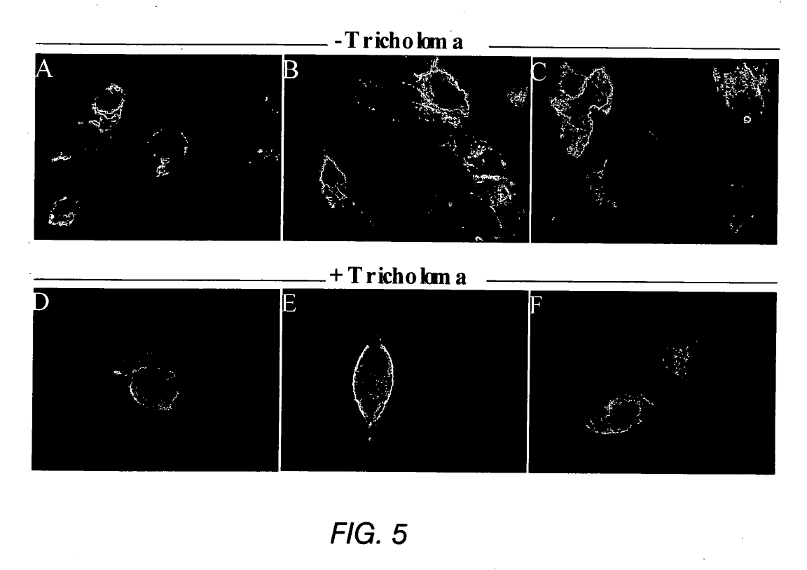

To determine whether the growth retardation of R6/GFP-Ras colonies exposed to Tricholoma filtrate is due to the suppression of Ras protein, protein extracts derived from the treated and untreated R6 and R6/GFP-Ras cells were examined for Ras protein expression using western blot analysis. Result showed that addition of Tricholoma filtrate to the cultures did not reduce the level of GFP-Ras fusion protein expression identified with either anti-Ras or anti-GFP antibody (FIG. 4). The same blot was hybridized with anti-actin antibody as a protein loading control. Aside from the protein level, Tricholoma treatment did not alter the normal subcellular localization of the GFP-tagged Ras protein, nor the transforming morphology of GFP-Ras cells as shown in FIG. 5. In that figure, the GFP-Ras proteins are correctly localized in the inner surface of the plasma membrane of the untreated (FIGS. 5A-C) as well as of the treated (FIGS. 5D-E) R6/GFP-Ras cells co-cultivated with R6 cells, as described in the experiments shown in FIG. 3C. No alteration of subcellular localization of GFP-Ras proteins was seen when R6/GFP-Ras cells were grown alone and treated with Tricholoma filtrate (data not shown).

3.6 The Inhibitory Effect of Gynostemma pentaphyllum Saponins Requires the Presence of Normal R6 Cells.

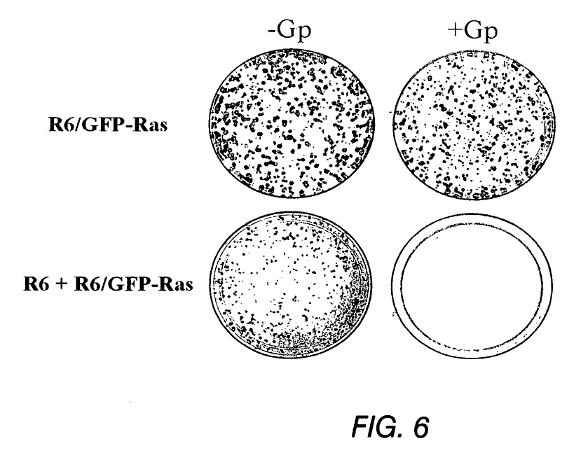

In previous experiments, we have demonstrated that Gp saponins do not impose a significant effect on either normal or the transformed R6 cells, rather the Gp saponins inhibit the formation of ras- and GFP-ras-induced foci. Our results suggested that the presence of R6 might play a role in the inhibitory effects of Gp. To explore the hypothesis, a co-culture assay which mimics the focus formation assay was designed. In that, the R6/GFP-ras transformed cells were seeded directly on monolayer R6 cultures with and without the addition of Gp saponins. The results are shown in FIG. 6. Colony formation of R6/GFP-ras cells was hardly affected by Gp treatment when the cells were cultured alone, but was markedly inhibited when R6/GFP-ras was co-cultured with R6 cells. Most of the R6/GFP-ras in the co-cultures remained as single cell emitting green fluorescent signal under the fluorescent microscope in the presence of Gp (data not shown). Further experiments showed that the inhibitory effect is dosage-dependent (data not shown). To test whether similar effects from Gp saponins would be seen when applied to human tumor cell lines, we tested an epithelial human bladder tumor cell line, T24. When T24 cells were used as a substitute for the GFP-ras transformed cells in the co-culture assay, a similar inhibitory effect was observed, namely, that Gp would only exert its inhibitory effect in the presence, but not in the absence of normal R6 cells in the same culture (data not shown). The inhibition of the colonies was also dosage dependent in the T24 cell experiment.

4. Discussion

In order to explore other possible cell-mediated responses to PS, we employed a non-lymphocytic in vitro cell system and tested the anti-tumor activity of PS against cell transformation induced by a defined ras oncogene. The results showed that PS from both the fruiting bodies and the cultured mycelia of G. lucidum markedly inhibited the formation of ras-induced transformed foci assessed by the focus formation assay in the R6 embryo fibroblast cell line. Interestingly, none of the PS preparations were toxic to either normal or ras-transformed R6 cells. Thus, the inhibitory effect of the mushroom extracts is not due to a direct cell killing of the transformed cells used in the study. This finding is consistent with the observations made in lymphatic cell system in which no cytotoxicity was detected under the treatment of G. lucidum (4,9,10).

The inhibitory effect was not restricted to G. lucidum. When we examined culture filtrate from Tricholoma, similar results were obtained. In the previous study, an anti-tumor component was identified in the culture filtrate of T. lobayense (17). The active component, based on tumorigenesis studies in animals, was found to reside in the DEAE-cellulose ion exchange column bound fraction, but not in the unbound fraction. Further characterization of the bound fraction showed that the fraction is a polysaccharide-protein complex with molecular weight of 154 kDa. It is intriguing that the levels of inhibitory activity of the DEAE-bound and -unbound fractions of Tricholoma assessed by focus formation assay were remarkably similar to those obtained by animal tumorigenesis test studied by Liu et al. (17)(Table VI).

TABLE VI
Comparisons of anti-cancer effects of
filtrates of T. lobayense cultures assessed
by animal test vs. in vitro focus formation assay

| | | Bald/c | Focus formation |
|---|---|---|---|
| | ICR male mice* | male mice* | assay |
| Material tested | (% of inhibition) | (% of inhibition) | (% of inhibition) |
| Crude extract | 84% | 66% | 90% (100 μg/ml) |
| DEAE-column | 96% | 50% | 96% (100 μg/ml) |
| Purified | | | |
| fraction (A2) | | | |
| DEAE- | 40% | 6% | 9% (100 μg/ml) |
| unbound | | | |
| fraction (A1) | | | |
| No treatment | 0% | 0% | 0% |

*Mice were injected intraperitoneally for 10 consecutive days with 20 mg of each tested samples/kg/day or distilled water as a negative control. Anti-tumor activities of each sample were tested against the growth of solid S-180 tumor transplanted in both ICR and Balb/c male mice [*animal data was obtained from Liu & Chang (17)]

The inhibitory activity of the bound fraction appeared to be dosage-dependent. Based on time course studies, early withdrawal of the component impaired the full activity of the PSPC, as shown in FIGS. 2B-D. On the other hand, a 32% inhibition was still observed when the addition of the chemical was delayed until day 12 (FIG. 2G). In fact, our study shows that the duration, rather than the time of application, dictates the efficacy of the compound, as demonstrated by the relative number of foci of Group B and H; C and G; and D and F. Each group of the pair received the same duration, but different time frame of treatment, yet each yielded a similar number of foci. We also observed that the relative foci decreased from 0.32 to 0.04 when the experiment was carried out for 24 (Table III), instead of 20 days (FIG. 2). This result reiterates the tentative conclusion that it is the length, not the time frame, of treatment that is more critical in determining the extent of inhibition of transformed foci.

The mechanism underlying the inhibitory effect of PS or PSPC against ras-induced foci remains unclear. In this study, the ras-transformed cells in focus formation were effectively inhibited during the early stage of transformation, and were equally inhibited when the stably transformed cells were mixed with normal cells, then treated with PS extract in the co-culture assay (FIG. 3C). Three key findings in this study may shed light to the possible mode of inhibition against ras-foci. Firstly, treatment with the extracts posts no cytocidal effect on either normal or established ras-transformed cell line assessed by the cell proliferation test and colony formation assay (Table II and FIGS. 3A, B). Secondly, mushroom extracts do not block the expression (FIG. 4), nor alter the membranous localization and the transforming activity of the ras oncoprotein tagged with GFP displayed in R6/GFP-ras cells (FIG. 5). Thirdly, the inhibitory effect of mushroom extracts against ras-transformed cells requires the presence of normal cells. The last point was well illustrated in the co-culture experiment, in which the colony formation of R6/GFP-ras cells was only inhibited in the present, but not in the absence of the co-cultured normal R6 cells (FIG. 3C). As mentioned earlier, treatment with Tricholoma does not affect cell adhesion as the number of seeded R6/GFP-Ras cells found in the treated cultures was similar to that found in the untreated cultures. Thus, the mushroom extract seems to exert its opposing effect on cell expansion, rather than on cell adhesion of the transformed cells. Early report indicated that certain triterpenoids from G. lucidum inhibited farnesyltransferase activity of Ras protein and retarded the growth of k-ras transformed cells (22). In our case, based on the clear display of the membraneous GFP-tagged Ras protein under the treatment with Tricholoma filtrate observed in vivo, the PS extract do not seem to act as farnesyltransferase inhibitor. Taking all these observations together, our data suggests that the anti-tumor effect of PS or PSCP from G. lucidum and T. lobayense is very likely mediated through the normal Rat 6 host cells, by direct or indirect cell contact. Based on our preliminary investigation, however, inhibition of the Tricholoma filtrate on the growth of transformed cells was not apparent when normal and transformed cells were each grown on an individual chamber (upper and lower) separated by a microporous membrane using a Transwell culture chamber system, suggesting that the inhibitory effect of mushroom filtrate may require a direct cell-to-cell contact (data not shown). However, determining the precise target of the PS and PSPC requires further investigation. Our previous works indicate that the transforming ability of the activated ras oncogene can be modulated by various factors and compounds (14-16). Early works by others suggested that the anti-tumor effect of fungal PS and PSPC is mediated through the cytokine released from the host cells. Later, Wang et al. presented evidence that treatment with G. lucidum stimulated macrophages and T lymphocytes to release TNF-α and IFN-γ, both of which were cytotoxic to HL-60 and U937 (9, 23). Other related studies that may shed light on the mechanism of mushroom extract are the recent works on glucan, a natural polysaccharide product widely distributed in fungi. Glucan has been reported to act as immunomodulator and cell response modifier. Binding of glucan to its specific glucan receptors can elicit a serial cellular response through the modulating of activities of various factors including IgE, cytokines, chemokines, transcriptional factors, and growth factors (24-26). Interestingly, the bioactive glucan receptors are present in human fibroblasts (26). Whether a similar mechanism applies to the inhibitory effect of mushroom extracts in our cell system warrants further investigation.

This study is the first to demonstrate that the PS and PSCP enriched mushroom extracts can inhibit cell transformation induced by a defined oncogene through a novel non-cytocidal route. It has also demonstrated the applicability of the system to testing of other, non-mushroom derived compounds and the use of other transformed cells in the assay, in particular a human cell line. Ras proteins play a pivotal role in regulating cell growth and the development of human cancer. The demonstration of the inhibitory effect of mushroom extracts on ras-induced transformation in this current study may have broad implications for cancer prevention and treatment and may provide a better understanding of the underlying mechanism of the cancer inhibitory effect of mushroom polysaccharides.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit and scope of that which is described and claimed.

### REFERENCES

1. Mizuno, T., Saito, H., Nishitoba, T., Kawagishi, H. (1995) Antitumor-active substances from mushrooms. Food Reviews International, 11,23-61.
   2. Miyazaki, T., Nishijima, M. (1981) Studies on fungal polysaccharides. XXVII. Structural examination of a water-soluble, antitumor polysaccharide of Ganoderma lucidum. Chem Pharm Bull. 29:3611-6.
3. Zhang, J., Wang, G., Li, H., Zhuang, C., Mizuno, T., Ito, H., Mayuzumi, H., Okamoto, H., Li, J. (1994) Antitumor active protein-containing glycans from the Chinese mushroom songshan lingzhi, Ganoderma tsugae mycelium. Biosci Biotech Biochem. 58, 1202-5.
4. Wang, H. X., Liu, W. K., Ng, T. B., Ooi, V. E. C., Chang, S. T. (1995) Immunomodulatory and antitumor activities of a polysaccharide-peptide complex from a mycelial culture of Tricholoma lobayense., A local edible mushroom. Life Science, 57, 269-281.
5. Yun, T. K., Kim, S. H., Lee, Y. S. (1995) Trial of a new medium-term model using benzo(a)pyrene induced lung tumor in newborn mice. Anticancer Research 15, 839-46.
6. Lu, H., Kyo, E., Uesaka, T., Katoh, O., Watanabe H. (2002) Prevention of development of N,N′-dimethylhydrazine-induced colon tumors by a water-soluble extract from cultured medium of Ganoderma lucidum (Rei-shi) mycelia in male ICR mice. International Journal of Molecular Medicine. 9,113-7.
7. Chen, T. W., Wong, Y. K., Lee, S. S. (1991) In vitro cytotoxicity of Ganoderma lucidum on oral cancer cells. Chung Hua i Hsueh Tsa Chih—Chinese Medical Journal, 48:54-8.
8. Kim, R. S., Kim, H. W., Kim, B. K. (1997) Suppressive effects of Ganoderma lucidum on proliferation of peripheral blood mononuclear cells. Molecules & Cells, 7, 52-7.
9. Wang, S. Y., Hsu, M. L., Hsu, H. C., Tzeng, C. H., Lee, S. S., Shiao, M. S., Ho, C. K. (1997) The anti-tumor effect of Ganoderma lucidum is mediated by cytokines released from activated macrophages and T lymphocytes. International Journal of Cancer, 70,699-705.
10. Sakagami, H., Aoki, T., Simpson, A., Tanuma, S. (1991) Induction of immunopotentiation activity by a protein-bound polysaccharide, PSK. Anticancer Research, 11,993-9.
11. Wang, Y. Y., Khoo K. H., Chen S. T., Lin, C. C., Wong, C. H., Lin, C. H. (2002) Studies on the immuno-modulating and antitumor activities of Ganoderma lucidum (Reishi) polysaccharides: functional and proteomic analyses of a fucose-containing glycoprotein fraction responsible for the activities. Bioorganic & Med Chem, 10, 1057-62.
12. Hsiao, W.-L. W., Wu, T. and Weinstein, I. B. (1986) Oncogene-induced transformation of rat embryo fibroblasts cell line is enhanced by tumor promoters. Mol. Cell. Biol., 6, 1943-1950.
13. Hsiao, W.-L. W., Lopez, C. A., Wu, T. and Weinstein, I. B. (1987) A factor present in fetal calf serum enhances oncogene-induced transformation of rodent fibroblasts. Mol. Cell. Biol., 7, 3380-3385.
14. Lopez, C. A., Hsiao, W.-L. W., and Weinstein, I. B. (1989a) Effects of various chemical agents on the transformation of rat fibroblasts by an activated c-Ha-ras oncogene. Molecular Carcinogenesis, 2, 81-87.
15. Lopez, C. A., Hsiao, W.-L. W., and Weinstein, I. B. (1989b) Effects of triiodothyronine (T3) and tamoxifen on cell transformation induced by an activated c-Ha-ras oncogene. Cancer Res., 49,895-939.
16. Hsiao, W. L. W., Pei, H., Matsui, M., and Weinstein, I. B. (1990) Effects of specific fatty acids on cell transformation induced by an activated c-H-ras oncogene. Oncogene, 5, 417-421.
17. Liu, F., Ooi, V. E., Chang, S. T. (1995) Anti-tumor components of the culture filtrates from Tricholoma sp. World J microbiology Biotechnology, 11, 486-490.
18. Freeman, A. E., Black, P. H., Vanderpool, E. A., Henry, P. H., Austin, J. B., and Huebner, R. J. (1967). Transformation of primary rat embryo cells by adenovirus type 2. Proc. Natl. Acad. Sci. U.S.A., 58, 1205-1212.
19. Bacchetti, S., Graham, F. L. (1977) Transfer of the gene for thymidine kinase to thymidine kinase-deficient human cells by purified herpes simplex viral DNA. Proc. Natl. Acad. Sci. U.S.A., 74,1590-4.
20. Wigler, M., Pellicer, A., Silverstein, S., Axel, R. (1978) Biochemical transfer of single-copy eucaryotic genes using total cellular DNA as donor. Cell, 14,725-31.
21. Lu, X., Park, S. H., Thompson, T. C. and Lane, D. P. (1992) Ras-induced hyperplasia occurs with mutation of p53, but activated ras and myc together can induce carcinoma without p53 mutation. Cell, 70,153-161.
22. Lee, S., Park, S., Oh, J. W., and Yang C. H. (1998) Natural inhibitors for protein prenyltransferase. Planta Medica 64, 303-308.
23. Lieu, C. W., Lee, S. S., Wang, S. Y. (1992) The effect of Ganoderma lucidum on induction of differentiation in leukemic U937 cells. Anticancer Research, 12,1211-5.
24. Brown, G. D., Gordon, S. (2003) Fungal beta-glucan and mammalian immunity. Immunity, 19, 311-5.
25. Lebron F., Vassallo, R., Puri, V. and Limper, A. H. (2003) Pneumocystis carinii cell wall beta-glucans initiate macrophage inflammatory responses through NF-kappaB activation. J Biological Chemistry, 278, 25001-8.
26. Wei, D., Williams, D., Browder, W. (2002) Activation of Ap-1 and SP1 correlates with wound growth factor gene expression in glucan-treated human fibroblasts. International Immunopharmacology, 2,1163-72.

1. A method for evaluating the ability of a substance to influence the rate of cellular proliferation comprising exposing a culture comprising transformed cells and non-transformed cells to said substance and determining whether said substance influences the proliferation rate of said transformed cells.

2. The method of claim 1, wherein said rate of cellular proliferation is measured in terms of a parameter selected from the group consisting of reduction in foci, reduction in number of cells, number of apoptotic cells, number of necrotic cells, measures of cell quiescence, changes in the intensity of a signal and the presence of a marker.

3. The method of claim 1, wherein said substance is selected from the group consisting of biological extracts, isolates, fractions, compounds, proteins, peptides, glucans, polysaccharides, polysaccharide protein complexes and inorganic compounds.

4. The method of claim 1, wherein said transformed and said non-transformed cells differ based on a characteristic selected from the group consisting of organism of origin, organ of origin, tissue or origin, clonal line, pre-experimental treatment, transfection with plasmid, exposure to a microorganism, infection with a virus and exposure to ionizing radiation.

5. The method of claim 1, wherein said step of determining whether said substance influences the proliferation rate of said transformed cells comprises comparing the proliferation rate of said transformed cells in said culture to the proliferation rate of transformed cells in a control culture.

6. The method of claim 5, wherein said control culture comprises transformed cells and nontransformed cells which have not been contacted with said substance.

7. The method of claim 5, wherein said control culture comprises transformed cells which have not been contacted with said substance.

8. The method of claim 1, wherein said step of determining whether said substance influences the proliferation rate of said cells comprises determining whether said substance decreases the proliferation rate of said cells.

9. The method of claim 1, wherein said culture comprising transformed cells and nontransformed cells is obtained by mixing transformed cells with nontransformed cells.

10. A method for evaluating the ability of a substance to influence the rate of cellular proliferation comprising:
contacting a culture comprising transformed cells with said substance and with culture medium from a culture comprising nontransformed cells; and
determining whether said substance influences the proliferation rate of said transformed cells.

11. The method of claim 10, wherein said transformed cells comprise a sequence that increases the proliferation rate of the cells, said sequence having been introduced to the cells by a method selected from the group consisting of stable transfection, transient transfection, random recombination, homologous recombination, exposure to a viral vector, exposure to ionizing radiation and chemical mutation.

12. The method of claim 11, wherein said transformed cells comprise sequence for expression of a marker molecule that can be used to distinguish said transformed cells from said nontransformed cells.

13. The method of claim 12, wherein said markers indicate expression of a protein, cell replication or release of compound into the media.

14. The method of claim 10, wherein said culture comprises fibroblast cells.

15. The method of claim 10, wherein said rate of cellular proliferation is measured in terms of a parameter selected from the group consisting of reduction in foci, reduction in number of cells, number of apoptotic cells, number of necrotic cells, measures of cell quiescence, changes in the intensity of a signal and the presence of a marker.

16. The method of claim 10, wherein said substance is selected from the group consisting of biological extracts, isolates, fractions, compounds, proteins, peptides, glucans, polysaccharides, polysaccharide protein complexes and inorganic compounds.

17. The method of claim 10, wherein said transformed and said nontransformed cells are cultured in the same media, separated by a permeable barrier or membrane.
